(19) United States
(12) Patent Application Publication
(10) Pub. No.: US 2022/0236800 A1
(43) Pub. Date: Jul. 28, 2022

(54) MAGNETIC BRAIN COMPUTER INTERFACE SURFACE MEMBRANE AND METHODS OF USING SAME

(71) Applicant: Austin Georgiades, Marlborough, CT (US)

(72) Inventor: Austin Georgiades, Marlborough, CT (US)

(21) Appl. No.: 17/390,541

(22) Filed: Jul. 30, 2021

Related U.S. Application Data
(60) Provisional application No. 63/069,046, filed on Aug. 22, 2020.

(51) Int. Cl. G06F 3/01; A61N 2/02; A61N 2/00

(52) U.S. Cl. CPC G06F 3/015; A61N 2/006; A61N 2/02

(57) ABSTRACT

A computer-brain interface includes a flexible substrate configured and arranged to be disposed within a subarachnoid space of a patient's head, and at least one layer having an electromagnetic coil array configured and arranged to measure and/or stimulate an activity of different regions of brain tissue that is capable of generating an action potential.

### CROSS-REFERENCE TO RELATED APPLICATIONS

This application claims priority to U.S. Provisional Ser. No. 63/069,046, filed on Aug. 22, 2020, the contents of which is hereby incorporated by reference in its entirety as if fully set forth herein.

### FIELD OF THE DISCLOSURE

The present disclosure relates to devices and methods for creating a brain-computer interface. More particularly, the present disclosure relates to a brain-computer interface having a plurality of electromagnetic coils.

### BACKGROUND OF THE DISCLOSURE

Brain-computer interfaces (BCI) are systems that provide communications between biological neural networks and some sort of electronic, computational device. BCIs can be used, for example, by individuals to control an external device such as a wheelchair using neural activity which is read from their brain. A major goal of brain-computer interfaces (BCIs) is to decode intent from the brain activity of an individual, and respond to said intent in a desired way. One example of the promise of BCIs relates to aiding people with severe motor impairments. However, conventional devices and methods are unnecessarily invasive and therefore limited in scope.

### SUMMARY OF THE DISCLOSURE

In some embodiments, a computer-brain interface includes a flexible substrate configured and arranged to be disposed within the subarachnoid space of a patient's head, and at least one layer having an array of electromagnetic coils configured and arranged to measure and/or stimulate activity in different regions of tissue that is capable of generating an action potential.

### BRIEF DESCRIPTION OF THE DISCLOSURE

Various embodiments of the presently disclosed devices and methods are described herein with reference to the drawings, wherein:

FIG. 1 is a simplified schematic representation of electromagnetic coils and the resulting magnetic fields;

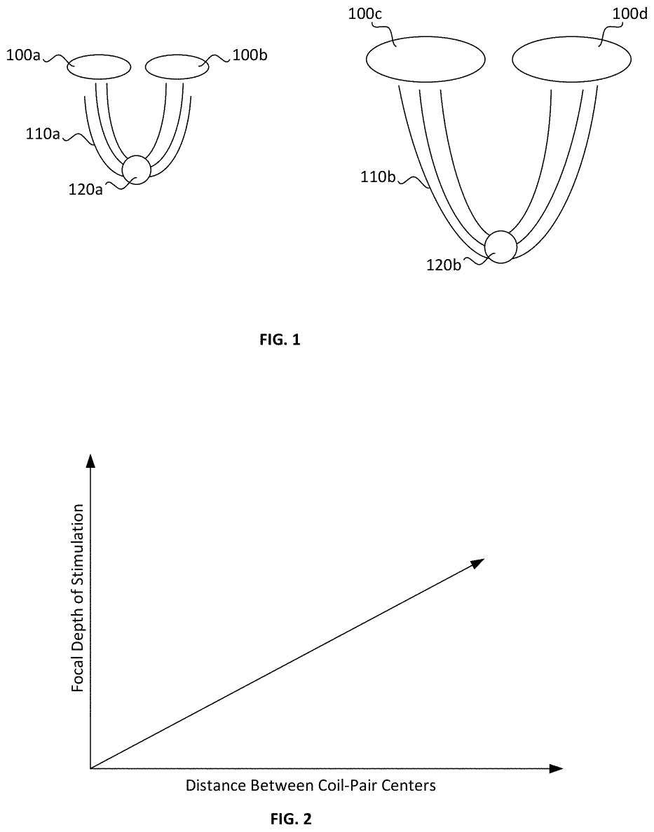

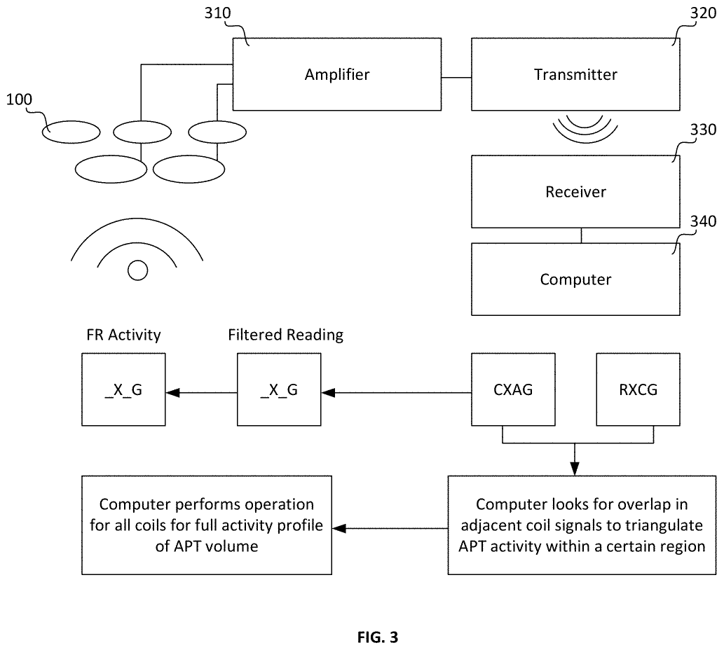

FIG. 2 is a simplified graph showing the general relationship between focal depth and distance between coil-pair centers;

FIG. 3 is a schematic representation of a system in reading mode, whereby the activity of APT can be determined using computational analysis of output signals;

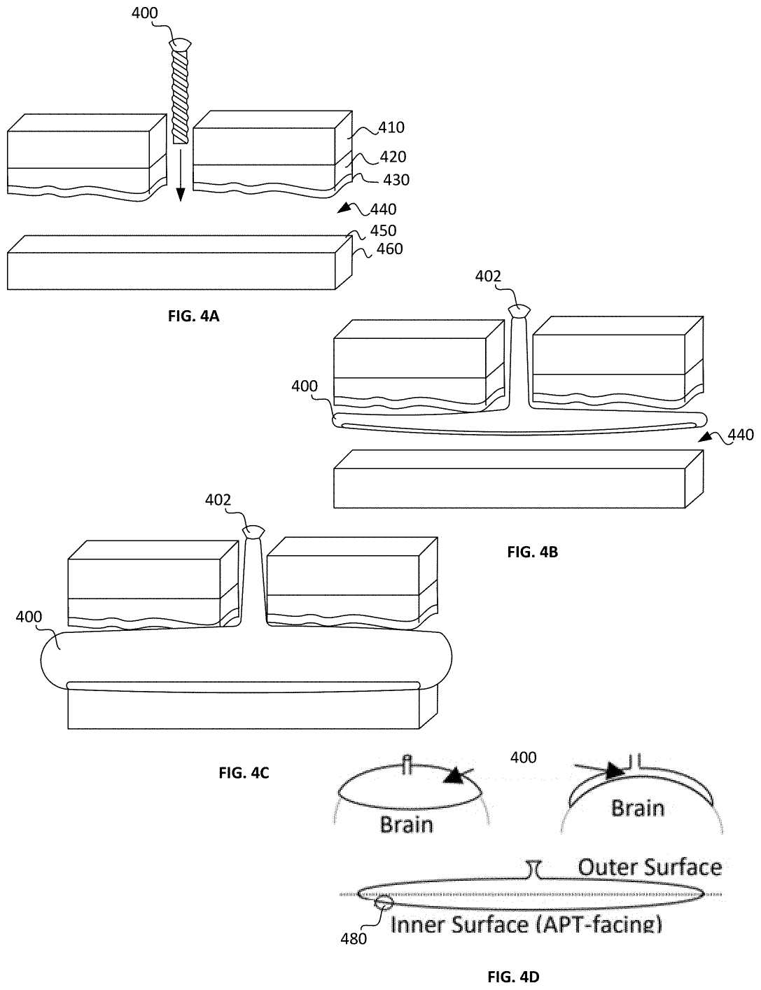

FIGS. 4A-D illustrate the use of a magnetic brain computer interface according to some embodiments of the disclosure;

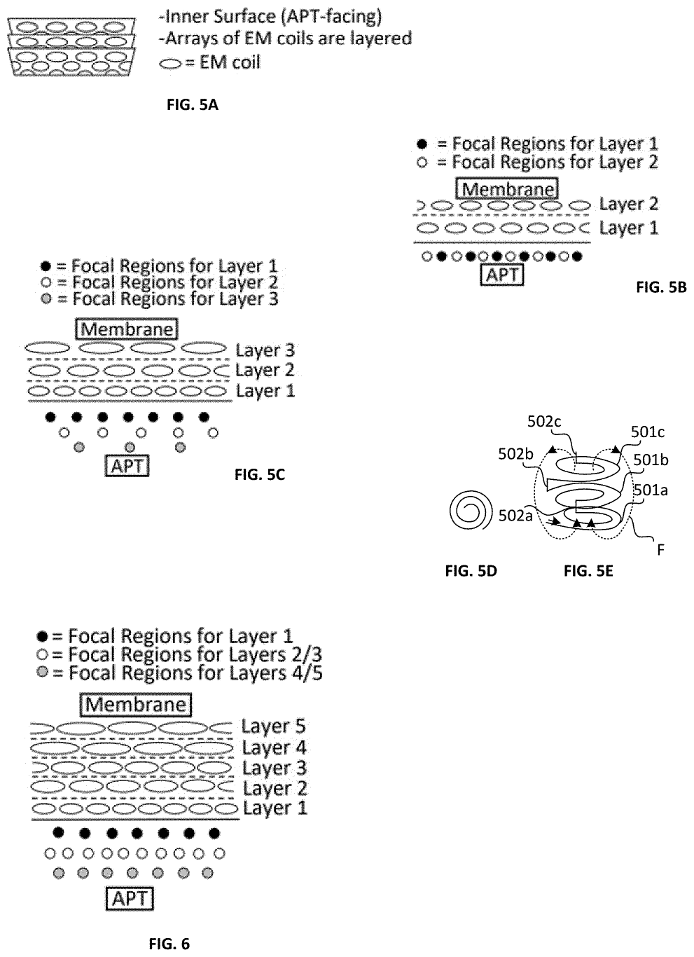

FIGS. 5A-E and 6 illustrate variations of the layers of the magnetic brain computer interfaces and how those variations relate to the focal regions; and

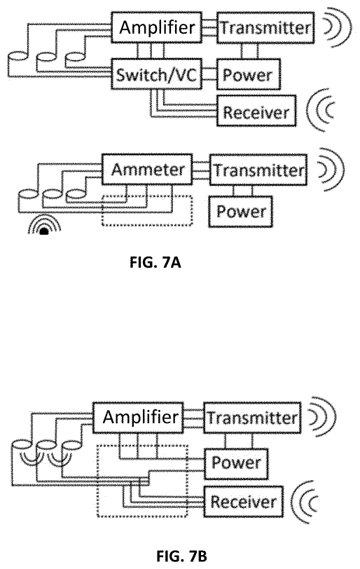

FIGS. 7A-B are schematic illustrations of a simplified circuit diagram in reading and writing modes.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to brain-computer interfaces (BCIs), conventional devices suffer from some shortcomings. Devices which measure and stimulate from outside the skull are significantly limited in resolution whereas current invasive BCIs require extensive surgical intervention to implant and are therefore limited in scope.

There therefore is a need for further improvements to the devices, systems, and methods of manufacturing and using brain--computer interfaces. Among other advantages, the present disclosure may address one or more of these needs.

As used herein, the term “proximal,” when used in connection with a component of a brain-computer interface, refers to the side of the component closest to or in contact with the brain when the brain-computer interface is implanted in a patient, whereas the term “distal,” when used in connection with a component of a brain-computer interface, refers to the end of the component farthest from the brain when the assembly is inserted in a patient.

In some embodiments, a flexible, artificial, circuit-membrane composed of layered, electromagnetic (EM) coil arrays designed to stimulate and measure the activity of different regions of tissue-capable-of-generating-an-action-potential (APT) and the necessary control and communications circuitry needed to make it a brain-computer interface (BCI). As used herein, an “APT” is defined as any number of cells greater than or equal to one which are capable of generating an action potential in response to an applied EM field of some orientation and magnitude. As used herein, an “action potential” is defined as an electrical current across a cell membrane which causes current to flow across nearby regions of cell membrane capable of conducting an electrical current. Moreover, “membrane,” as defined herein, comprises any broad, relatively thin, surface or layer. When stimulating, it should be noted that the current passing through EM coil pairs may be flowing in different directions, thus creating two magnetic dipoles pointed in opposing directions when said coils are coplanar.

According to some embodiments, the present disclosure may utilize similar underlying principles as transcranial magnetic stimulation (TMS). In TMS, EM coils (also referred to as inductors) of conductive wire are arranged to stimulate regions of brain tissue from outside the skull using a dynamic magnetic field to induce a current in the target region of brain tissue above the threshold required to induce an action potential, thus inducing an action potential in neural tissue. The surface position, magnetic field strength, coil geometries and relative angles between the two coils determine the three-dimensional flux distribution of the applied magnetic field. The region between two non-overlapping, coplanar, dynamic, magnetic dipoles of equal and opposite magnitude in a uniformly conducting medium which has a magnetic flux density large enough, given a certain rate of change in the magnetic field strength, to induce an action potential in APT shall henceforth be referred to as the focal region. For any non-zero, dynamic, magnetic field, if the magnitude of the rate of change in the magnetic field is increased from zero until a single contiguous region of APT is stimulated between the two coils, it is this focal region which will contain the point of greatest distance beyond the plane of the coils that is above the threshold of stimulation. This point of furthest distance from the plane of the coils which is above the threshold of stimulation shall henceforth be referred to as the focal point. In the case that the applied magnetic field is not strong enough to form a single, contiguous region above the threshold of stimulation between two coils, two focal points are formed. Thus, coils can be used to target point-like regions of space within specific layers of neural tissue to stimulate by positioning the focal point at the depth of the layer being targeted for stimulation.

EM coils, when arranged in series with a current monitor, may also be used to measure changes in EM potential. The ability for an EM coil to measure changes in the surrounding EM field depends upon changes in the EM field inducing a current in the coil, which is measured by the current monitor (measuring coil). The magnitude of induced current in a measuring coil depends on both the magnitude of the change in the EM field as well as the location of the source of said change relative to the measuring coil(s). As a rule, the relative strength of the current induced in a measuring coil is inversely proportional to the square of the distance the source of the current fluctuation is away from the measuring coil, is proportional to the magnitude of the source current fluctuation and is trigonometrically proportional to the angle between the plane of the coil and the source of the current fluctuation. This means that EM field fluctuations positioned closer to a measuring coil will induce a greater magnitude of current in the coil than EM field fluctuations of equal magnitude positioned further away from and at the same angle relative to the plane of the coil, EM field fluctuations of greater magnitude will induce more current in the measurement coil than those of lesser magnitude in the same location, and sources of EM field fluctuations positioned at a greater angle relative to the plane of the coil will induce more current than an equivalent source of EM field oscillations at an equal distance but lesser angle. In order to locate EM field changes of unknown magnitude within a region of three-dimensional space, a minimum of four non-coplanar measuring coils of known location may be used. In a process called true-range multilateration, the relative amplitudes of a signal (as compared across the readings of four non-coplanar coils of known location) can be used to locate the source of said signal within three-dimensional space. Layering multiple two-dimensional arrays of measuring coils allows for the three-dimensional location of signals within a volume of APT to be measured. Being able to specify the 3-D locations of APT activity over time means a BCI employing layered arrays of measuring coils can provide a 4-D map of activity within a volume of APT.

By arranging EM coils in arrays across the surface of APT and controlling the current that flows through them, the number of distinct regions of tissue that can be stimulated may increase. Additionally, or alternatively, by arranging multiple layers of EM coil-arrays across the surface of APT, the volume of tissue that can be accurately measured may increase (along with the potential precision of the measurements). The systems necessary to individually control EM coil activity, including controlling current strength and direction, as well as controlling the switch between active-stimulation-mode and passive-measurement-mode may be incorporated into circuitry. Coordinated, higher control over these individual EM coil functions would enable stimulation of many different discrete regions of APT and allow accurate measurement of activity across a volume of APT. Thus, the device may function as a BCI.

For example, to further increase the number of discrete, point-like regions that can be stimulated, EM coil arrays can be stacked on top of each other to form a three-dimensional, laminar structure called a coil matrix. EM coil size, geometry, spacing between coil centers and angle relative to surface may all be modified between layers and/or within layers and/or not at all, if necessary, to produce the desired distribution of focal points within the target tissue. For example, EM coils with greater spacing between their centers have focal points further away from the plane of the coils. Thus, EM coils with greater spacing between their centers may be used to stimulate deeper regions of APT. EM coils located within the same layer of membrane can stimulate an array of focal points at a certain depth when paired only with coils immediately adjacent, but can also stimulate deeper regions of APT as well by pairing with non-adjacent coils further away. The magnitude of current which must pass through non-adjacent coils would need to be larger than it would be in the case of adjacent coils in order to stimulate the pair's focal point. Being able to vary the magnitude of current supplied to coils enables arrays of focal points at different depths to be stimulated from a single layer of EM coils.

In theory, the number of distinct focal points that can be stimulated by an array of electromagnetic coils is equal to the number of unique combinations of coil-pairs which can be formed. In practice, the inability to pass enough current through coils to stimulate their respective focal point when separated by a large distance may prevent the full potential of total possible focal points from being stimulated. For any given reference coil, there is a certain radius within which combinations of coils (which include the reference coil) are capable of stimulating their respective focal points. Working to increase the range of current which can be passed through coils extends the maximum distance between coils in which stimulation of the focal point is still possible. In addition, the focal region stimulated can be enlarged by increasing the current flowing through any EM coil-pair beyond what is required for threshold stimulation of the focal point, thereby increasing the volume of tissue where stimulation is above the threshold required to produce an action potential. Thus, the volume of the region stimulated can be modulated by varying current. The range of volume that can be stimulated by a coil-pair depends on the range of current which can be supplied to coils. Coil-pairs which are further apart have less dynamic range in the amount of volume they can stimulate beyond the formation of the first single focal point than coil-pairs which are closer together.

In theory, EM coil arrays do not need to be static nor laminar. For example, it is possible to have a dynamic, laminar model consisting of a grid of squares with 4-way junctions at each of the vertices. Control circuitry would require a means of directing how current flows through vertex-junctions in addition to controlling current strength and direction. The ability for dynamic junctions to control the circuit of the EM coil means that a grid of square electromagnetic coils can produce more possible circuits (and therefore magnetic fields and therefore unique focal regions) than the elemental square units that comprise it. Going even further, near the theoretical extreme, a cubic matrix with 6-way junctions at the vertices could even further increase the number of possible circuits which could be generated from a given membrane. A membrane such as this would constitute a non-laminar, dynamic model. In addition, or alternatively, it is possible to use the same set of coils to both measure and stimulate by switching between passive read mode and active stimulation mode versus using separate coil sets to measure and stimulate.

In some embodiments, a brain-computer interface device referred to herein as a “Magnetic Brain Computer Interface Surface Membrane” (MBCISM), may include a flexible, circuit-matrix containing EM coils on the side facing the APT and the necessary control systems required to: generate and/or store and manage power for the system, apply a variable voltage across coil circuits in both directions, passively measure induced current in EM coils, transmit coil measurement data to an outside device for recording/analysis and receive signals from an outside device which control stimulation. These supporting control systems can be incorporated into the flexible circuit wherever space allows. Power for the device can come by external, inductive charging or by an internal power supply mechanism, such as that described in U.S. patent application Ser. No. 17/069,867, which is incorporated herein by reference.

While the interface device may be used on all APT, it is optimized for use as a BCI. Its design allows for easy implantation and removal, such as that described in Magnetic Brain Computer Interface Surface Membrane Injector, Application: 63/069,307 (filed Aug. 24, 2020)). Unlike electrodes, there is no need to avoid blood vessels and nerves of APT during implantation, as nothing is physically disturbing, invasively contacting or impaling the APT. However, perhaps the greatest benefit of MBCISM with regards to serving as a BCI is the ability for the flexible circuit-membrane to cover a large surface area of brain and read/write across a large volume of APT with less surgical intervention required per unit volume of tissue with which it is interfaced than other invasive BCIs currently in the prior art. The circuit's flexibility combined with the specific shape of the membrane (much like a balloon) allows it to be slipped through a hole in the skull much smaller in area than the region of APT it covers once it is expanded within the subarachnoid space (or another internal cavity) to cover areas of APT beyond the peripheries of the hole it was inserted through. Just as an example, an ideally-wrapped flexible BCI 1/16 of a millimeter thick could cover a circular area of APT 48 mm in diameter through a 7 mm hole or a 90 mm diameter circular area through a 9 mm diameter hole. A BCI 1/32 of a mm thick could cover a circular area of APT 96 mm in diameter through a 7 mm hole and 160 mm in diameter through a 9 mm hole. This is compared to electrode-based, invasive BCI models that must remove skull equal to or greater in area than the entire area of APT they are to interface with. The theoretical size that circuit components may be printed into flexible circuitry suggests the ability to support a high density of coils within a single layer of flexible, circuit membrane (let alone multiple layers) and the theoretically large number of viable coil pairings which can be formed from among this potentially massive array highlight the capacity for a BCI of this design to read/write to and from APT with unparalleled bandwidth.

Specific methods for implantation such as inflation or expansion of a cavernous MBCISM within the subarachnoid space allow a large area of APT to be in contact with the proximal read/write surface of the membrane using a relatively small hole in the skull. This ability to significantly reduce the surgical intensity of implantation per unit of surface area of brain interfaced-with, while providing potentially equal, if not better, read/write resolution than electrodes (for which one would have to cut out a section of skull at least equal in area to the region of brain being interfaced with), provides the MBCISM with significant advantages as a BCI paradigm versus current electrode-based BCIs. Constant output of brain-state data from measurement across the potentially large volume of neural tissue the device is interfaced with can be used to gather a large amount of detailed information about cognitive processes. The possibility of covering the entire surface of the brain promises the ability to record the complete state of a person's brain at any time after implantation. This data can then be analyzed computationally and used to generate input which modulates brain activity. The input space is maximized by the large number of different focal regions within APT that can be stimulated. Thus, the MBCISM is optimized for use as a BCI.

FIG. 1 is a schematic representation of electromagnetic coils and the resulting magnetic fields. As illustrated in FIG. 1, two pairs of electromagnetic coils 100a-d and a portion of their respective magnetic fields 110a-b are shown in the case that the coil-pair receive current that passes in opposite directions to each other (so as to produce magnetic dipoles which are opposite in direction). The regions 120a-b represent the point (focal point) most distal to the plane of the EM coils that is above the threshold of stimulation if the focal region was just able to form one continuous volume versus two discrete volumes. If one were to increase the magnitude of change in the magnetic field from zero given a certain magnetic field strength, the first region of APT to reach stimulatory threshold is within the region of highest magnetic flux density. This demonstrates the principle by which a discrete region of APT can be stimulated by a dynamic magnetic field formed between two EM coils.

FIG. 2 is a simplified graph showing the general relationship between depth of focal points and distance between coil-pair centers. Specifically, FIG. 2 illustrates the general relationship between the separation between coil-pair centers and the distance of the focal point for said coil pair from the plane of the coils. This distance is termed “focal depth”. As shown, there is an expected possibly linear increase in the depth of the focal region as coil-pair center separation increases. Thus, this graph illustrates how APT can be stimulated at various depths by EM coils with varying distances between their centers.

FIG. 3 is a schematic representation of a system in reading mode, whereby the activity of APT can be determined using computational analysis of output. First, activity within APT induces current in EM coils 100. This is amplified by an amplifier 310 and the current values are transmitted via a transmitter to an outside receiver 320,330 which is connected to some kind of device 340 for storage and/or analysis. For simplicity, the illustration in FIG. 3 shows this process within two dimensions using only two coils, with the source located at equal distance from both coils shown. This computer system compares the amplified induced current readings from the coils to look for signal overlap and differences in signal amplitude. In two dimensions, signals which are equal in amplitude across the readings of two adjacent coils can be located along a line which is perpendicular to the plane of the coils. In three dimensions, these signals would exist on a plane which is perpendicular to the line segment between the two coil centers, which passes through the midpoint of said line segment.

Examination of the relative strengths of signals across four or more non-coplanar coils allows localization of the signal course within three-dimensional space through a process known as true-range multilateration. Multilateration uses the distances between an object and points of known location to calculate the spatial position of the object. Signal amplitudes measured by coils provides information about the distance a signal source is from points of known location and can thus be used to position signal sources within three-dimensional space. In this example, signals are represented as strings of letters, with the same letters indicating the same signal being detected by both coils and their position left to right representing their occurrence within time. Non-similarities represent signals above a certain threshold picked up by one coil at a certain time but not the other. In this way, discrete signals detected by multiple coils can be filtered to provide a map of activity occurring near the relevant coils.

FIGS. 4A-D illustrate the use of an MBCISM 400 according to one embodiment of the disclosure. Specifically, computer interface 400 may be introduced through a hole formed in skull 410 and dura mater 420 below the arachnoid mater 430 into the subarachnoid space 440 above pia mater 450 and facing brain 460. In this example, computer interface 400 is spirally wrapped on itself to collapse it into a delivery condition. Once situated at least partially within the subarachnoid space 440, the computer interface 400 may be unraveled, unfurled or otherwise expanded into the delivered condition shown in FIG. 4B. In some examples, the computer interface 400 may be inflatable and may be transitioned into a balloon-shaped or bladder-shaped inflated condition shown in FIG. 4C by delivering fluid or a gas through an inlet 402.

FIG. 4D is a perspective illustration showing what a cavernous MBCISM would look like when in operational contact with an exposed brain, resting much like a deflated whoopee cushion with an opening in the center adjacent to the APT it interfaces with. The cross-sectional illustration demonstrates a hollow interior with a face in contact with the APT surface (proximal) and a face which is directed away (distal). Control, communication and power systems may be located on the distal surface while electromagnetic coil arrays capable of reading and/or writing may be disposed on the proximal surface. The small, hollow protuberance on the top of the MCBISM may be used for the implantation and sub-cranial inflation and/or expansion of the device within subarachnoid space (for more detailed information on the mechanics of insertion via subarachnoid inflation, see: Magnetic Brain Computer Interface Surface Membrane Injector, Application: 63/069,307 (filed Aug. 24, 2020)). The interface 400 is shown as being open and laterally-extending in this illustration. It should be noted that, after implantation, the opening of the MBCISM may be sealed and the interior volume may be filled with a fluid so it bridges the gap between the pial surface of the brain and the arachnoid surface of the interior face of the skull. The MBCISM may be pressed against both surfaces with enough pressure to ensure conformation to the pial surface of the brain & to brace the device against any shifts in position which may occur post-implantation, thus ensuring the best possible interface across the entire proximal surface. One of the key advantages of MBCISM 400 is that the flexible membrane can be inserted through a relatively small hole in the skull and then inflated and/or expanded to cover an area much larger than the hole through which it is inserted, thus simplifying the implantation operation versus electrodes over an equivalent area, reducing risk, scarring and recovery time. In FIG. 4D, a section of the proximal surface of the MBCISM is circled with reference 480 and variations of this section are enlarged in FIGS. 5A-E and 6.

FIGS. 5A-E and 6 illustrate variations of the layers of coil arrays in the MBCISM and how those variations relate to the distribution of focal points within the APT. Specifically, the illustration in FIG. 5A shows a magnified cross-section of a portion of the proximal side of the MBCISM, showing layered arrays of EM coils (shown as ovals) that together form a composite matrix. In FIG. 5B, there is illustrated one of the various ways that coil-layers can be arranged to achieve certain results. For example, multiple layers (e.g., two layers) of EM coils (with the coils being laterally offset) that share the same focal depth for increased read/write density or resolution at that depth. Coil layers within the circuit membrane are labeled and their respective focal points within APT are shown. It should be noted that the solid line separating the coil layers from the focal regions demarcates the transition between MCBISM and APT and the dashed line is used to demarcate the boundary between separate coil-layers. FIG. 5C shows multiple layers (e.g., three layers) of EM coils with focal points trained to different focal depths for measurement and simulation across different depths of APT. In this example, layers may have different focal depths (e.g., three separate focal depths) to measure/stimulate multiple depths of APT.

Each of the EM coils 501a-c may include layers of spirals of conductive material similar to that shown in the top view of FIG. 5D. Layers may include alternate coils 501a-c spiraling in and out of layered planar surfaces to produce a net magnetic field “F” through the center of all coils (FIG. 5E). Links 502a-c between layers (shown as vertical lines) are also possible. Layers may be separated by a minimum distance (e.g., 1 or more polymer length).

The illustration in FIG. 6 shows how coil-layers of varying lateral offsets and focal depths can be arranged in many ways to create a custom distribution of focal points at varying depths of APT. It also highlights the fact that focal depth can be almost continuously varied to create an ideal volumetric distribution of focal regions. This custom distribution of focal regions can be optimized for various APT to create the best possible interface for a target tissue. Although the focal points shown are only those formed by pairing between adjacent coils, it should be noted that other focal regions can be stimulated too via pairing of non-adjacent coils (which includes coils within the same layer as well as non-adjacent coils within different layers). In addition, although each coil pair is shown as having one focal point in this illustration, the size of the focal region may be variable for coil pairs, meaning the focal region may be shifted deeper by increasing stimulatory drive beyond the minimum threshold required to form a single focal point. Increasing the magnitude of change in the current passing through a coil pair (and thus increasing the strength of the current induced by the field) beyond what is required for this threshold stimulation causes the region of APT being stimulated by a coil pair to increase in volume & depth, thus the volumetric profile of focal regions can be varied as well and focal regions of different sizes may be mixed-and-matched.

FIGS. 7A-B are schematic illustrations of a simplified circuit diagram in reading and writing modes. This series of illustrations show a simplified circuit diagram of a MBCISM to illustrate how reading and writing occur. The illustration of FIG. 7A represents a simplified circuit diagram, showing three EM coils connected to both an amplifier (also described as an ammeter) and a voltage controller/switch. The amplifier is connected to a transmitter, which sends readings wirelessly to an outside device using energy from the power supply. The voltage controller/switch, meanwhile, is connected to the power supply and a receiver. The receiver gives the voltage controller/switch signals as to what electromagnetic coils to send current to, the magnitude of the current being sent and the direction of the current.

When reading (FIG. 7A), a switch simply bypasses all of the voltage source and closes the circuits for the EM coils. Circuits for all coils run through an amplifier, so current induced in the EM coils by APT activity can be measured and transmitted to an outside device. It should be noted that not all circuitry within the voltage controller (dashed box below the amplifier) is shown here. Instead, only relevant circuits are illustrated for the sake of simplicity. The function of MBCISM circuitry with regards to reading is to collect the greatest number of coil-readings possible from across as wide a volume of APT as possible, with the highest possible resolution for induced-current measurements.

When writing (FIG. 7B), the receiver sends signals to the switch to connect certain electromagnetic coils to the voltage controller. For simplicity, the control of current from the power source to various coil circuits is illustrated in the diagram by the points of contact between the output from the receiver and the power supply to the coils. However, the receiver not only controls which coils have current running through them, but also how much current flows and in what direction. Circuitry responsible for controlling current magnitude and direction is not shown explicitly in the diagram, but occurs within the voltage-controller/switch complex with input from the receiver. Gating of current may occur via transistors controlled by the receiver. The writing function of MBCISM circuitry is to operate as a system capable of inducing action potentials in as many variably-sized, discrete regions of APT as possible within a set amount of time. The determination of which coil-pairs should be stimulated, how much current should be sent through each coil pair, and the direction of current that passes through each coil is made by an outside computational device. The receiver is simply responsible for executing commands sent from an outside device via its input to MBCISM control circuitry.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

We claim:

1. A computer-brain interface, comprising:
a flexible substrate configured and arranged to be disposed within a subarachnoid space of a patient's head; and
at least one layer having an electromagnetic coil array configured and arranged to measure and/or stimulate an activity of different regions of brain tissue that is capable of generating an action potential.

2. The computer-brain interface of claim 1, wherein the flexible substrate comprises a polymer.

3. The computer-brain interface of claim 1, wherein the flexible substrate comprises an inflatable balloon having a collapsed condition for delivery and an expanded condition during use.

4. The computer-brain interface of claim 1, wherein the flexible substrate comprises a proximal surface and a distal surface, the at least one layer being disposed on the proximal surface.

5. The computer-brain interface of claim 4, further comprising a controller being disposed on the distal surface.

6. The computer-brain interface of claim 1, wherein the at least one layer comprises multiple layers, and wherein the electromagnetic coil array comprises a three-dimensional electromagnetic coil matrix.

7. The computer-brain interface of claim 6, wherein the multiple layers comprise two layers.

8. The computer-brain interface of claim 6, wherein the multiple layers comprise three layers.

9. The computer-brain interface of claim 6, wherein the multiple layers comprise a first array of electromagnetic coils being disposed on a first layer, and a second array of electromagnetic coils being disposed on a second layer, the first array and the second array being laterally offset.

10. The computer-brain interface of claim 6, wherein the multiple layers comprise a first array of electromagnetic coils being disposed on a first layer, and a second array of electromagnetic coils being disposed on a second layer, the first array and the second array being laterally aligned.

11. The computer-brain interface of claim 6, wherein the multiple layers comprise a first array of electromagnetic coils being disposed on a first layer, and a second array of electromagnetic coils being disposed on a second layer, the first array and the second array having a same focal depth.

12. The computer-brain interface of claim 6, wherein the multiple layers comprise a first array of electromagnetic coils being disposed on a first layer, and a second array of electromagnetic coils being disposed on a second layer, the first array and the second array having multiple focal depths.

13. The computer-brain interface of claim 12, wherein the multiple focal depths comprises two focal depths.

14. The computer-brain interface of claim 12, wherein the multiple focal depths comprises three focal depths.

15. The computer-brain interface of claim 12, wherein the first array of electromagnetic coil comprises non-adjacent electromagnetic coils being paired together.

16. A method of forming a computer-brain interface, comprising:
forming a flexible substrate;
forming at least one layer on the flexible substrate, the at least one layer having an electromagnetic coil array configured and arranged to measure and/or stimulate an activity of different regions of brain tissue that is capable of generating an action potential; and
delivering the flexible substrate within a subarachnoid space of a patient's head.

17. The method of claim 16, wherein forming a flexible substrate comprises forming a balloon-shaped substrate.

18. The method of claim 17, further comprising delivering the balloon-shaped substrate in a collapsed condition.

19. The method of claim 17, further comprising inflating the balloon-shaped substrate to an expanded condition.

20. The method of claim 17, wherein forming at least one layer comprises forming multiple stacked layers on the flexible substrate to create a matrix of electromagnetic coils.
